(19) United States
(12) Patent Application Publication
(10) Pub. No.: US 2005/0202436 A1
(43) Pub. Date: Sep. 15, 2005

(54) Target-specific multiple sequencing primer pool for microbial typing and sequencing applications in DNA-sequencing technologies

(76) Inventor: Baback Gharizadeh, Stockholm (SE)

Correspondence Address: Baback Gharizadeh, 321 Hawthorne Avenue, Palo Alto, CA, 94301, US

(21) Appl. No.: 10/623,458

(22) Filed: Jul. 17, 2003

Related U.S. Application Data
(60) Provisional application No. 60/399,357, filed on Jul. 30, 2002.

(51) Int. Cl. C12Q 1/70; C12Q 1/68; C07H 21/04

(52) U.S. Cl. 435/6; 536/24.3; 435/5

(57) ABSTRACT

The invention relates to a method of typing a sample of nucleic acid molecules in DNA sequencing technologies, whereby the molecules are suspected to comprise at least DNA from one type or species, e.g. microorganisms or viruses, with each species or type or target DNA having different nucleotide patterns. By adding a mixed set of at least two oligonucleotide primers, whereby each primer is designed for being specific for one type or species or target DNA, thereby allowing a primer or primers to hybridize in or close to the type-specific or target regions; determining the types or targets sequence by extending the hybridized primer or primers in a sequencing reaction. Hereby, the typing is simplified by using a set of type-specific primers and sequence pattern recognition approach. The method of invention is suitable for samples containing a plurality of types or species, multiple pathogenic infections or variants, and amplicons with unspecific amplification products. Furthermore, the invention relates to a kit for use in the method.

Note: This patent application is related to the provisional patent application with the application No. 60/399,357 filing date Jul. 7, 2002.

### STATEMENT REGARDING FEDERALLY SPONSORED RESEARCH OR DEVELOPMENT

The invention is not federally sponsored.

### Abstract

The invention relates to a method of typing a sample of nucleic acid molecules, whereby the molecules are suspected to comprise at least one type/species/target DNA of a type-specific/target region chosen from a known set of types of the type-specific/target region, each species/type/target having different nucleotide patterns, comprising the steps of: providing the sample of nucleic acid molecules; providing a mixed set of at least two oligonucleotide primers, whereby each primer is designed for being specific for one type/species/target DNA chosen from the known set of types of the nucleic acid sample, thereby allowing a primer, which is specific for a type that is present in the sample, to hybridize in or close to the type-specific/target region; mixing the sample and the set of primers under conditions allowing a primer to hybridize to the sample if a type/species/target that is specific for the primer is present in the sample; determining the type/target sequence to which a primer has hybridized by extending the hybridized primer in a sequencing reaction. Hereby, by using a set of type-specific/target primers the typing is simplified. The method of invention is suitable for samples containing a plurality of types/species, multiple infections/variants and amplicons with unspecific amplification products. Furthermore, the invention relates to a kit for use in the method.

### TECHNICAL FIELD

The invention relates to a method of specific genotyping/typing/detection/identification/sequencing of a sample of nucleic acid molecules by DNA sequencing, where one (two or more in case of plurality of genotypes/species in the sample) of the specific oligonucleotides binds to the target. The multiple sequencing oligonucleotide primer approach could be used for different purposes. Target specific multiple sequencing primer pool applications are:
1. Specific detection and identification of amplicons containing multiple variants/infections amplified with consensus/degenerate primers, containing more than one genotype as in human papillomavirus (HPV), whereby the molecules are suspected to comprise at least one type of a variable region chosen from a known set of types of the variable region, each type having different nucleotide patterns. The sample may be from a microorganism, such as a multiple infected sample. This approach is illustrated in FIG. 1.
2. Specific detection of genotypes/target DNA of interest with designed type-specific oligonucleotides in amplicons amplified with consensus/degenerate primers or multiplex PCR, amplifying more than one genotype/type/species (e.g. in human papillomavirus detecting clinically important or high-risk oncogenic genotypes of interest routinely or identification of relevant bacterial species). This approach is illustrated in FIG. 2a, showing specific detection of HPV-16 and HPV-18 in two different samples and FIG. 2b demonstrating target specific detection of two bacterial species by multiple sequencing primers amplified by multiplex PCR.
3. Target specific detection of genotypes of interest in amplicons containing unspecific amplification (e.g. using general/degenerate primers for PCR amplification, which might amplify other regions of the genome especially in one step PCRs such as amplifying with GP5+/GP6+ or MY09/11 consensus primer sets in human papillomaviruses). Utilizing multiple sequencing primer pool eliminates the need for nested PCR or cloning. This approach is illustrated in FIG. 3. The sample may contain both unspecific amplification products and multiple infections as illustrated in FIG. 1. Additionally, FIGS. 4a and 4b show unspecific amplification from genomic DNA on agarose stained gel by GP5+/6+ and MY09/11 amplification products in clinical samples, which can be clearly sequenced and genotyped correctly with the method of invention but not with the general/PCR primer.
4. Winning read-length by circumventing semi-conservative regions. FIG. 5 illustrates principally specific detection of amplicons amplified with general/degenerate primers containing semi-conservative regions as in bacteria where semi-conservative sequencing regions could be skipped, allowing faster sequencing and covering more DNA length of target/variable region due to read-length limitations of DNA sequencing techniques. This enhances the sequence accuracy and quality. FIG. 6 shows this principle on clinical 16S amplicons.
5. Amplicon(s) with low yield or amplicon(s) containing a sub-dominant type, which could not be detected due to low sequence signals when in presence of the dominant sequence signals (unspecific amplification or multiple types/species sample). FIG. 7a shows a clinical sample with extremely low sequence signals, which cannot be detected by the general sequencing primer due to unspecific amplification and is detected by multiple sequencing primer. FIG. 7b shows a clinical sample with low PCR yield with multiple infections sequenced by general primer and multiple sequencing primer pool.

The invention could be tailored and adapted according to the desired applications or clinical settings. Furthermore, the invention relates to a kit for carrying out the method of the invention.

### BACKGROUND OF THE INVENTION

In any genome, several kinds of genetically important variations or allelic variations occur. These can be insertions, deletions, single nucleotide polymorphisms (SNPs) or short tandem repeats (STRs).

In order to determine what type of variation that is present in a nucleic acid sample typing is performed. Typing refers to any method for analyzing the nucleotide sequence of a nucleic acid molecule. Specifically, typing methods include methods for detecting, identifying or analyzing genetic or sequence variation in one or more target nucleic acid(s). Consequently, genotyping refers to the determination of the genotype of the target nucleic acid molecule. The genotype, in turn, refers to a combination or pattern of multiple genetic variations (or “variable sites”) in the target nucleic acid.

Typically, typing may be performed by using conventional DNA sequencing techniques. Dideoxy (Sanger) sequencing (Sanger et al., 1977), Pyrosequencing (Ronaghi et al., 1998) and Mass Spectrometry DNA Sequencing (Berkenkamp et al., 1998) or any other DNA-sequencing technique applicable to this technique could be used. When using a sequencing technique in a normal way for genotyping a sample, an oligonucleotide primer is designed, where the primer is able to hybridize to the single stranded form of the sample close to the variable site or region of interest in question. Hereby, the primer is extended over the variable site or region of interest during sequencing, and thus the nucleotide pattern of the variable site or region of interest is determined, i.e. the sample strand is typed.

Also, various hybridization techniques may be used for typing, DNA sequencing methods are more reliable than hybridization techniques (which are more based on signal detection only), since sequencing techniques provide nucleic acid information, which are the core of every organism. Thus, DNA-sequencing techniques are preferred for typing technologies of the field of the invention.

However, in some cases a sample may comprise more than one type/species of a variable site of interest. This is the case for a multiple infected sample, such as a sample of HPV. For HPV a number of types are known, which may be divided in two groups: high-risk and low-risk types. The high-risk types have been recognized as causative agents for cervical cancer and are related to other types of cancer. Using a known approach (Gharizadeh et al.), a primer extendable over the variable site and hybridizing in proximity to the variable site is allowed to hybridize to the sample of interest. The primer is then extended and information concerning the sequence of the variable site is revealed. However, if several types are present in the samples, hence comprising different nucleotide patterns at the variable site, the risk that the sequence information is difficult or impossible to interpret is high, since signals from all the available types will be produced. This is a result of that the primer binds to all present genotypes of the sample (FIG. 1).

In another case, PCR primers and general/consensus primers in PCRs (especially in one-step PCRs) may yield unspecific amplification products due to different reasons in amplification process. When using the general PCR primer as sequencing primer, the general PCR primer will hybridize to all present specific amplified products as well as the unspecific amplified products, giving rise to mixed sequence signals from specific and unspecific PCR amplifications in the sequencing process, making genotyping impossible (FIGS. 1 and 3).

Another significant and interesting application of the target-specific multiple oligonucleotide pool is for specific detection/identification/typing/genotyping of specific types of interest in microorganisms and viruses by DNA sequencing such as in human papillomaviurses; HPV-16 and HPV-18 are the most high-risk genotypes that are clinically important and many laboratories test primarily for these two high-risk genotypes. Thus, with a pool of two specific primers for HPV-16 and HPV-18, one can run a one-step-PCR (eliminating nested PCR or cloning) and genotype only these two types regardless of other available genotypes or unspecific amplifications (FIG. 2a). If both genotypes are present in the sample, pattern recognition (alignments of sequence signals from both genotypes) could be used for genotyping of both HPV-16 and HPV18. Another example is genotyping of Chlamydia trachomatis and Neisseria Gonorrhea amplified by multiplex PCR. They could be both detected by using the method of invention by using a pool of two oligonucleotides, each oligonucleotide specific for one species (FIG. 2b). Based on this, species-specific oligonucleotide primers for species of interest in microorganisms and viruses could be designed in relevant diagnostic kits according to required criteria such as geographic regions or other clinical and research purposes with different amplification procedures. Other examples for the method of invention are typing of different genotypes of Hepatitis B virus, Rotavirus and Chlamydia.

Pourmand et al (Pourmand et al., 2002) discloses the use of a multiplex Pyrosequencing approach. Hereby, the nucleotide identity of several different variable sites is possible to detect in one single species/type. This document also discloses the use of their approach for microbial typing of hepatitis C virus. This method is based on hybridizing a pool of three oligonucleotides to one species/type (HCV) on three different regions on the same type (not different types). The primers are extended simultaneously on the same fragment by nucleotide addition. This will result in three sequence signals, which should be calculated for HCV-typing. The idea behind this invention is shorten read length in DNA sequencing. By obtaining sequence from three regions one type/species simultaneously, and calculating the signals one can faster genotype HCV.

However, as mentioned above and as disclosed in Gharizadeh et al (Gharizadeh et al., 2001), multiple infections and unspecific amplifications present in one specimen is problematic to detect. The target specific multiple sequencing oligonucleotide pool approach enables skilled person to easily type samples comprising a plurality of types/species or when the amplicon contains unspecific amplification. Multiple sequencing primer approach provides sequence data of high quality because of the specificity of the primers without interference from unspecific amplification products. This will also eliminate use of specially treated PCRs such as nested PCR or re-performing PCRs or cloning.

DNA sequencing is not very sensitive to simultaneously detect different sequences in a sample. Sequences representing a minority of total PCR product may remain unnoticed and only the dominant genotype will be detected. By utilizing multiple specific sequencing primers, species/genotypes of low dominance will be detected (FIG. 7a).

PCR products with low yield will be difficult to detect due to low signals when using the general primers as signals from unspecific amplification products or multiple infections interfere and the signals from the relevant genotype is not noticed. Multiple sequencing primer method detects fragments of low yield (FIG. 7b).

In the case, where the primers in the primer pool are annealed to more than one genotype/species/target, the pattern recongnition will be used for detection of the genotypes/species. The pattern recognition is based on the sequence characterisics of each species/target by the sequence alignments (FIG. 8).

Thus this is an object of the invention to provide a method, which satisfies these needs. DNA sequencing is the gold standard method. It is more reliable than the hybridization techniques, which are based on signal detection. DNA sequencing provides nucleic acid information, which is the core of every organism.

### BRIEF SUMMARY OF THE INVENTION

These and other objects are accomplished by a method for typing a sample of nucleic acid molecules, whereby the molecules are suspected to comprise at least one type of a variable/target region chosen from a known set of types of the variable/target region, each type having different nucleotide patterns comprising the steps as defined in claim 1 of the invention. The amplicon might also contain unspecific amplification, giving rise to sequence signals making base calling difficult or impossible. The method could also be used for direct typing of species/genotypes of interest. It could as well be used for detection of species/genotypes of low PCR yield or sub-dominant types/species.

Hereby, by providing a mixed set of oligonucleotide primers to the same sample only the primers having a complementary nucleotide sequence in the sample will hybridize, and thus only the type(s) related to these primers will result in sequence signals. Accordingly, this method is highly advantageous when analyzing a sample comprising several types of one variable/target site, such as a multiple-infected sample.

There are other regions on the human papillomavirus genome (E6 and E7), where type-specific PCR is used for detection of high-risk HPV genotypes such as HPV-16, HPV-18 and HPV-33 (Skyldberg et al., 1991; Hagmar et al., 1995). These type-specific PCRs could be performed in multiplex, amplifying the genotype (instead of performing single PCR) and a designed oligonucleotide pool could be used for detection by DNA-sequencing technologies.

According to the method of the present invention a pool of two or more primers, each specific for a specific type of, for example, a virus, bacterium, fungus or a target DNA, is added to a sample to be analyzed for the presence of the different type(s) presumably present in the sample. If DNA complementary to one of the primers is present, the primer will hybridize to this DNA and function as sequencing primer allowing the type-specific DNA to be sequenced. The method of the present invention therefore allows the distinction of closely related, but still different, types of, for example, bacteria, fungi and viruses.

Some of embodiments for which the primer set chosen according to the present invention that are favorable are:
1. Specific detection and identification types/target of related DNA-types, such as clinically relvant types of a bacterium, fungi, virus or other target DNA, present in a sample (FIG. 2).
2. Specific detection and identification types/target of related DNA-types, containing multiple variants/infections, such as clinically important types of a bacterium, fungi, virus or other target DNA, present in a sample (FIGS. 1, 8 and 9).
3. Specific detection and identification of a genotype/type in a sample that contains unspecific amplification products, avoiding the problem of unspecific amplification products interfering with the specific sequencing results, (FIGS. 1, 3 and 10). The need for nested PCR, cloning or re-performing of failed PCRs is eliminated.
4. Detection of types/genotypes/targets/species in PCR products with low yield or when the type of interest is sub-dominant (FIG. 7).
5. Winning read length by circumventing to sequence semi-conservative regions. This will result in faster DNA sequencing with improved quality (FIGS. 5 and 6).

In another aspect of the invention a kit of oligonucleotides is provided for performing the method of the invention. The kit comprises the contents as defined in the claims and is especially adapted for performing the method of invention.

In an additional important aspect, the invention can be used for direct and specific sequencing/identification/detection/typing/genotyping of species or subspecies of interest in microorganisms. Kits can be developed for performing the method of the invention. By using the concept of the invention when typing a nucleic acid sample comprising at least one type of a type-specific/variable/target region chosen from a known set of types, or a plurality of types of the variable region as in the case of a multiple-infected sample, high-quality typing results are achieved.

To summarize (FIG. 1-12), the new method is especially suited for detection and typing of samples harboring different DNA targets, such as multiple infected/variant/species/types samples, or specific genotyping of clinically relevant targets/types/species/groups of interest, or when the sample containing unspecific amplifications. The method is applicable to samples with low PCR yield or samples containing subdominant types/species. The method of invention can also be used for winning read-length.

The new method was used in a model system for detection and typing of high-risk and clinically relevant low-risk human papillomaviruses (HPV) in multiple infections and samples containing unspecific amplification products. Type-specific sequencing primers were designed for five of the most oncogenic (high-risk) HPV-types (HPV-16, HPV-18, HPV-31, HPV-33 and HPV 45). The primers were combined and added to a sample containing a mixture of one high risk (HPV-16, 18, 31, 33 and 45) and one or several low-risk types. The primed DNA samples were sequenced by Pyrosequencing technology and dideoxy DNA-sequencing. In all combinations tested correct genotyping was possible. The multiple oligonucleotide approach improved the sequence data quality for samples containing unspecific amplication. A seven-oligoculeotide pool (specific for HPV-6, 11, 16, 18, 31, 33 and 45) was used for 80 clinical samples and very good results were obtained on samples with multiple infection.

The new method was also used for faster and more specific typing of bacteria. Bacterial DNA was amplified with PCR utilizing general primers binding to conservative regions on the 16S rRNA gene. The bacterial amplicons used in this study could be grouped in two categories, one group with 19 bases sequence similarity downstream of the PCR primer and the second group with 31 bases sequence similarity. Two sequence-group specific sequencing primers were designed and added together to the amplified bacterial DNA. Fast and correct typing of bacteria from both groups was possible by the Pyrosequencing technology. The method was applicable to Sanger DNA sequencing as well. The new method is especially useful in combination with the Pyrosequencing technology (this method for the moment is limited to sequencing about 50-100 bases depending on the template) as the method of the present invention is not dependent on starting the sequencing in a conserved region of the DNA. Thereby less of the number of bases that can be amplified using the Pyroseuqnecing technology are “wasted” on sequencing conserved regions and more of the number of bases that can be sequenced are actually used for sequencing of the variable regions.

The multiple sequencing oligonucleotide method can in a totally different approach be used for quantification of at least two different DNA-types in a sample provided that the DNA-sequence is known and two primers bind at the same time. The sequence signals of both amplicons can be compared together for quantification.

The method of invention is applicable to Sanger DNA sequencing method, Pyrosequencing technology and it is presumed to work with mass spectrometry DNA-sequencing.

### BRIEF DESCRIPTION OF THE FIGURES

FIG. 1 shows the principle of typing of human papillomavirus (HPV) in mixed infections/variants using a general primer (FIGS. 1a and 1c) and the primer set of the invention (FIGS. 1b and 1b).

FIG. 2a shows the principle of specific genotyping of clinically important human papillomaviruses (HPV-16 and HPV-18) according to the method of invention. FIG. 2b illustrates the principle of typing two bacterial species amplified by nested PCR; pattern recognition is used for typing when both species are present.

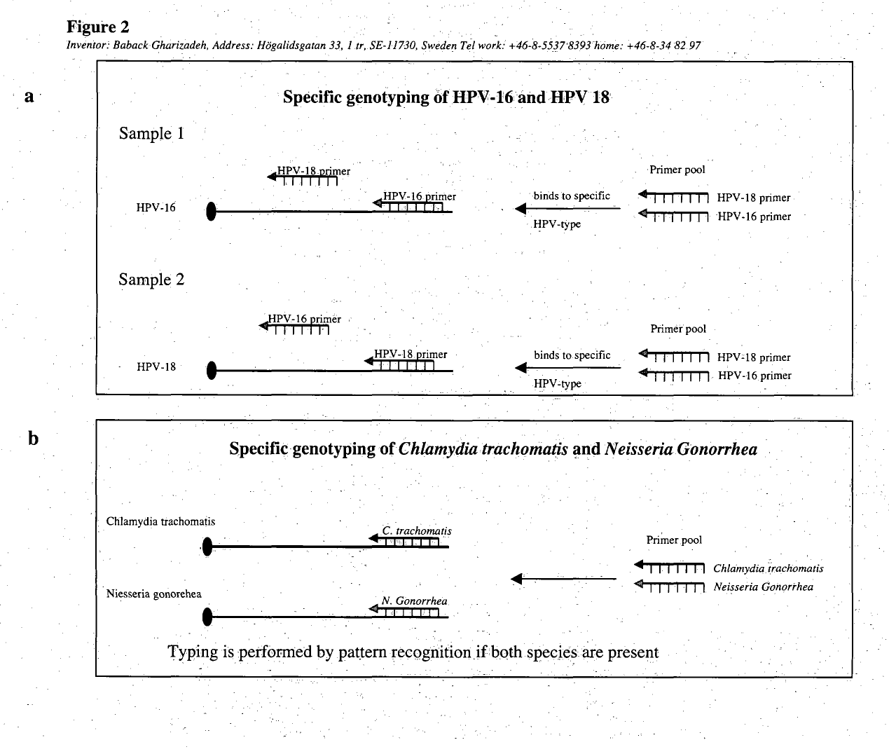

FIG. 3 shows the principle of typing amplicons containing unspecific amplification products (a) sequenced by the general primer and (b) sequenced by the type-specific multiple sequencing primer pool of the invention.

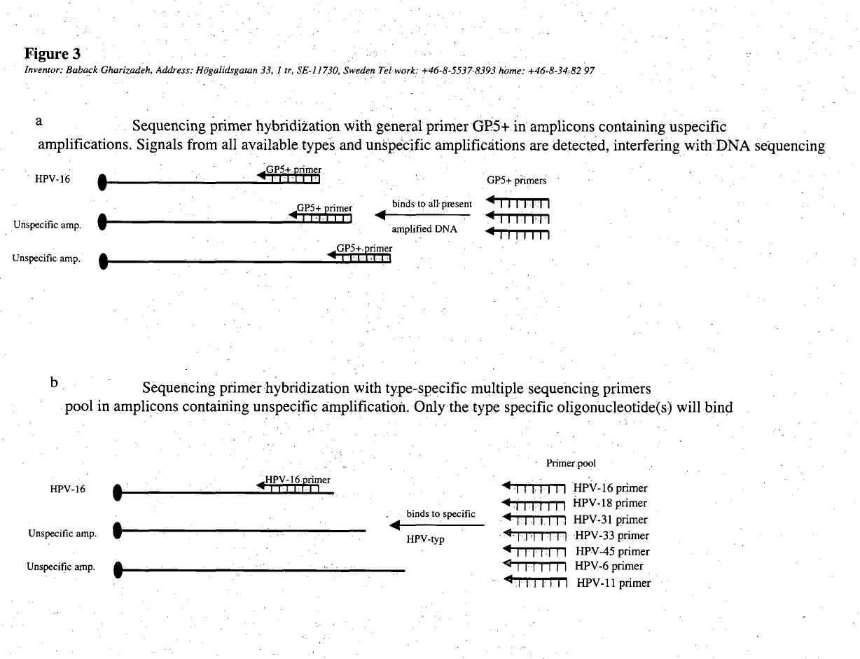

FIG. 4 shows unspecific amplification on clinical samples from genomic DNA on ethidium bromide agarose stained gels by (a) degenerate primers MY09/11 and (b) general (consensus) primers GP5+/6+

FIG. 5 shows a schematic illustration of the principle winning read length. a) Sequencing with the U3R general amplification primer. b) Sequencing with group-specific multiple-sequencing primer pool (Seq-19b and Seq-31b), where sequencing of 19 or 31 bases are circumvented.

FIG. 6 shows pyrograms of (a) Escherichia coli sequenced with general primer (b) Escherichia coli sequenced by primer set of invention skipping 31 bases of semi-conservative region with improved sequence quality(c) Streptococcus pneumoniae sequenced by general primer (d) Streptococcus pneumoniae sequenced by primer set of invention circumventing 19 bases.

FIG. 7 shows genotyping samples containing dominant multiple infections and unspecific amplification products a) amplicon containing unspecific amplification sequenced by general primer GP5+ b) sequencing of the same amplicon by multiple sequencing primers. HPV-16 is extremely sub-dominant, still being genotyped by in spite of extreme low yield c) amplicon containing unspecific amplification or multiple infection, sequenced by general primer GP5+ d) Sequencing of the same amplicon by multiple sequencing primers. HPV-33 could be easily detected in spite of low PCR yield

FIG. 8 shows genotyping of multiple infections of HPV-16 and HPV-18 in three clinical samples utilizing seven multiple sequencing primers. Genotyping is performed by pattern recognition. Genotyping by pattern recognition. The common and specific bases for each type is noted on top of each peak, characterizing each type, which facilitates genotyping. The dominant type could be easily observed by comparison of single bases shown by arrows. a) HPV-16 and HPV-18 almost equal in dominance b) HPV-18 dominant c) HPV-16 dominant

FIG. 9 shows pyrogram from sequencing of samples with multiple HPV variants with GP5+ primer (a, c, e, g), resulting in sequence signals from all present genotypes making genotyping impossible, and with (b, d, f, h) the type-specific four-seqencing-primer pool (for high-risk HPV-16, 18, 33 and 45), resulting in specific sequence signals of the clinically relevant high-risk HPV-types.

FIG. 10 shows pyrograms from sequencing of a clinical sample amplified by GP5+/6+ consensus primer set in a one-step-PCR and sequenced with a) GP5+ primer, yielding unspecific sequence signals, and with b) the type-specific four-seqencing-primer pool (for high-risk HPV-16, 18, 33 and 45); clear sequence signals were obtained and the sample was genotypes as HPV-16.

FIG. 11 shows electrophoregram of dideoxy sequence of a sample with multiple HPV variants (HPV-16/72/6) sequenced with a) GP5+ primer, showing sequence data from all present types, making genotyping impossible, and with b) the type-specific four-seqencing-primer pool (for high-risk HPV-16, 18, 33 and 45). Clear sequence data was obtained from the medically important HPV; the sample was genotyped as HPV-16.

FIG. 12 shows Sanger dideoxy electropherogram of of Listeria monocytogenes sequenced with the sequence-group specific seqencing-primer pool (Seq-19b and Seq-31b)

### DETAILED DESCRIPTION OF THE INVENTION

By a “variable region” in the context of this invention is meant a stretch of nucleotides within a genetic material, which shows a varying sequence between different nucleic acid molecules covering the same gene(s) or the same genetic part of the genetic material. A “conservative region” is essentially identical in different nucleic acid molecules. By a “type” is meant a specific variant (or nucleotide pattern) of the variable region. By “degenerate primers” is meant a mixture of similar primers that have different bases at the variable positions. By a “consensus or general primer” is meant a primer that binds to a conservative region. By a “multiplex PCR” is meant when more than two primers are used in the amplification process. By a “multiple infections” is meant when there are more than one variant/type/genotype/species of the microorganism(s) or virus(es) present in the sample. By “pattern recognition” is mean comparison/alignment of at least two sequence-pattern result (such as a pyrogram) from the same sample and determine the characteristic sequence for each type/species/group.

The present invention provides a method for typing a sample of nucleic acid molecules, which sample is suspected to contain at least one type/species/groLip-specific region chosen from a known set of types/species/groups of the specific regions. Thereby, the method according to the present invention allows the detection and identification of related, but still different, nucleotide types present in a sample. In short, according to the method of the present invention, a sample of nucleic acid molecules and a mixed set of at least two oligonucleotide primers, directed to type-specific regions, are provided. The sample and the primers are then mixed which allows the primer to hybridize to the sample DNA if complementary sequences are present. The hybridization primers function as sequencing primers allowing a sequencing reaction to be carried out and the identity of nucleic acid sequences to be determined.

In a first aspect, the invention relates to a method for typing a sample of nucleic acid molecules, whereby the molecules are suspected to comprise at least one type/species/genotype of a target/variable region chosen from a known set of types of the type-specific/variable/target region, each type having different nucleotide patterns, comprising the steps of:
Providing the sample of nucleic acid molecules
Providing a mixed set of at least two oligonucleotide primers, whereby each primer is designed for being specific for one type/species/group chosen from the known set of types of the nucleic acid sample, thereby allowing a primer (or primers in case of plurality of species/types), which is specific for a type that is present in the sample, to hybridize in or close to the target/type-specific/variable region.
Mixing the sample and set of primers under conditions allowing each primer to hybridize to the sample if a type that is specific for the primer is present in the sample
Determining the type/sequence to which a primer (or primers in the case of multiple infections or plurality of species) has hybridized by extending the hybridized primer(s) in a sequencing reaction.

The primer/oligonucleotide pool used to perform the present invention is designed especially for each sample type, depending on what sequences might be present and what sequences one wants to detect. The oligonucleotide primer pool to be used for the present invention comprises at least two primers, wherein each primer is directed to a different, target/type-specific sequence in the target/type-specific/variable region of the different types that might be present in the sample. Oligonucleotide primers can be designed for specific regions of different microorganisms and viruses, which makes the typing faster and more reliable.

By using a primer directed to a conserved region, as is done in the prior art, it is impossible to distinguish between closely related, but still different types present in a sample, since all the variants present will be sequenced. In comparison, by using a primer set according to the present invention, only the types for which a complementary primer (or primers) is (are) present will be sequenced and detected. Thereby the typing process is simplified and speeded up. Specific primers could be designed, which facilitates typing by shorter DNA reads. In the cases of presence of two or more (geno)types, a sequence pattern recognition will be applied for detection.

The method could be used for amplicons containing unspecific amplification products, amplicons with low yield or amplicons containing sub-dominant type. These advantages eliminate the need for nested PCR or cloning or re-performing PCRs. Another advantage of using a primer pool directed to variable regions according to the present invention, is avoidance of having to sequence non-variable, and therefore not type-specific DNA in order to reach the variable regions which have to be sequenced in order for a typing to be performed. DNA-sequencing technologies have limitation in reading length (number of the nucleotides that is possible to sequence from the position of the sequencing primer). Then, if a sequencing primer is designed for a conservative region, and the semi-variable region is positioned between the conservative region and the type-specific region of interest, the sequencing data from the type-specific region may be poor. However, if the sequencing primer, as in the invention, is designed for hybridizing to the semi-conservative region, the primer attaches closer to the target/group/type-specific region of interest. Then, sequences comprising less informative data need not be read, and the sequencing data of the specific region will be obtained faster with higher sequence quality.

Generally, the method is carried out in the following way: To a nucleic acid sample, preferably single-stranded DNA, a pool (set of primers) of two or more oligonucleotides is added, which are specific for a certain type of the sample material. The sample material may be of microorganisms or viruses. The oligonucleotide hybridizing to the nucleic acid molecule functions as a sequencing primer for a subsequent sequencing reaction. If the sample comprises a plurality of types in the range of the olignucleotide primers applied, the typing will be done by patter recognition. The pattern recognition could be simplified by finding a sequence characteristic for each type when primer designing.

In one embodiment of the invention, the method is used for a sample comprising several genotypes of a virus, such as human papillomaviruses, (in practice, it would not be known what genotypes are present in the sample). In this case, if the oligonucleotides of the set of primers of the invention are designed to only hybridize to the diagnostically interesting oncogenic high-risk genotypes, at least one of the oligonucleotides will hybridize to the sample and will at sequencing give a characteristic sequence. If the same sample comprises more than one high-risk genotype of interest, this sample will also be detected by the method by pattern recognition. In this latter case, at least quantitative information (amount and types of high-risk virus in the sample) will be possible to achieve.

If the number of types is too many for pattern recognition, this in itself indicates that there is more than one high-risk genotype (or species of interest), which is of clinical importance. In an approach, a separate sequencing of the sample with each primer individually could be performed. For example if a sample contains HPV-16, HPV-18, HPV-31, HPV-33, HPV45 and HPV-6 and HPV-11 and they are all detected by the seven-specific multiple sequencing primers. Sequence signal data from all these HPV types may be too many for pattern recognition. For specific genotyping (if not recognizable by sequence signal patterns), by applying each sequencing primer in a separate sequencing reaction, one can genotype all the present HPVs specifically as a solution. Although a in multiple pyrogram pattern that all the types cannot be genotyped because of simply presence of many genotypes (which is rare) is of clinical relevance (as all the primers are specific for clinically and medically important types). There are kits available (Hybrid Capture II, Digene Inc) only informing the presence of a low-risk or high-risk HPVs in the sample with no regard about the specific genotype.

Accordingly, in one embodiment the sample is suspected to comprise at least two types chosen from the known set of types. In yet anther embodiment the sample is a multiple infected sample. In still another embodiment at least one primer is specific for a high-risk variant of a disease linked to the infectious microorganism. In yet another embodiment the nucleic acid molecules are of microorganisms and viruses. Other application areas are not excluded.

There are over 100 known HPV-types. Of these a few are regarded as high-risk causing cervical cancer and head and neck cancer. HPV-16 is responsible for 50-60 percent of cases and HPV-18 for 10-20 percent. HPV-31, HPV-33 and HPV-45 are each accountable for approximately 5 percent each depending on demographic factors and regions. The low-risk HPV types stand for lower risk for cervical cancer meaning that they are not as effective as the high-risk. HPV-6 and HPV-11 are important in the low risk group for skin and genital warts. The known clinically important high- and low-risk HPV are
High-risk: 16, 18, 31, 33, 35, 39, 45, 51, 52, 58, 59, 66, 68, 69
Low-risk: 6, 11, 34, 40, 42, 43, 44

Accordingly, in one embodiment the known set of HPV set of HPV-types are chosen from the group comprising the HPV-types (high-risk) 16, 18, 31, 33, 35, 39, 45, 51, 52, 58, 59, 66, 68, 69 and (low risk) 6, 11, 34, 40, 42, 43, 44

In another embodiment the method according to the present invention is used for bacterial typing, wherein the type-specific region is specific for a group of bacteria. Primarily, the bacteria are chosen from clinically important groups of bacteria, such as Listeria species (such as Listeria moncyogenes), Staphyloococcus species (such as Staphyloococcus aureus, Staphyloococcus haemolyticus, Staphyloococcus pneumoniae), Streptococcus species (such as Streptococcus agalactiae, Streptococcus anginosus, Streptococcus intermedius, Streptococcus milleri, Streptococcus mitis, Streptococcus pneumoniae), Haemophilus species (such as Haemophilus influenzae), Neisseria species (such as Neisseria meningitidis), Enterococcus species (such as Enterococcus fecalis, Enterobacter cloacae) Escherichia coli and Klebsiella pneumoniae. However, the list above is merely examples of clinically important groups/species of bacteria, and the invention is applicable also for other bacteria. A pool (set of primers) of several oligonucleotide primers is added to a nucleic acid sample, preferably single stranded DNA, whereby only one kind (at most) of the oligonucleotides will hybridize to the sample, and thereby have the capacity to function as a sequencing primer (in Pyrosequencing) or a extension primer in dideoxy seqeuncing. The primer that hybridizes will be specific for a semi-conservative region specific for this group of bacteria. By a “semi-conservative” region is hereby meant a region, which distinguishes between different groups of bacteria. Yet, further differences may occur within the group (the group may comprise several types), which requires the need for a variable region to be analyzed. One advantage with this aspect of the invention is to get longer into the gene (compared to when using a general primer) in order to get sequencing data from the more variable region. Bacteria may be divided in different groups, and for each group, group-specific (in the bacterial experiment presented here, grouping was performed based on sequence similarity) primers are designed. Using this strategy, one does not need to (which is the case when using a general primer) read through stretches of less informative sequence.

Normally, this is a problem when analyzing variable regions with conventional sequencing technologies as they have a limitation in their reading length (number of nucleotides that is possible to sequence from position of the extension or sequence primer). Then, if an sequencing primer is designed for a conservative region, and a semi-variable region is positioned between the conservative region and the variable region of interest, the sequencing data from the variable region may be poor. However, If the sequencing primer, as in the invention, is designed for hybridizing to the semi-conservative region, the primer attaches closer to the variable region of interest. Then, sequences comprising less informative data need not be read, and the sequencing data of the variable region will be of better quality and the process is more time-effective. This applies for the entire DNA sequencing technologies as they have DNA read-length limitations.

The sample of nucleic acid molecules may be of any kind, such as DNA or RNA, and of any species origin. Preferably, the sample is of a microorganism, such as virus, bacteria and fungi.

The nucleic acid sample may beneficially be provided by isolation and purification of the sample. These steps may be performed by any conventional technique, which are known for a person skilled in the art. During amplification, the nucleic acid molecules may be amplified with biotin-labeled primers, or the like, in order to make it possible to bind the nucleic acid molecules to a solid phase prior to typing. This could be also performed on double stranded DNA as explained in (Nordstrom et al., 2000a; Nordstrom et al., 2000b)

The set of primers comprises at least two oligonucleotides. There is no limit in the number of primers. The upper limit is determined if interferences or complications are appeared for any reason. The primers might hybridize to each other, giving false sequence signals. However, this may be avoided by using single strand binding protein (SSB), or extra wash (to remove the non-hybridized primers) or due to a clever choice of the sequences of the primers.

In order for a primer to be specific for a certain type of the sample it is desirable that all nucleotides of the primer are complementary to the type-specific region of the type it is supposed to be specific for. It varies from case to. case how many nucleotides of the primer that are allowed to not base-pair to the sample in order a sufficient hybridization no to occur. Generally, about 2-3 mismatches are usually enough to prevent a sufficient hybridization to take place. However, It is most important that the extendable end (the 3′-end) of the primer hybridizes to the sample molecule, since otherwise extension may not be achieved.

If a primer is mis-hybridized (in extreme cases) in the hybridization step of the invention, binding to a different region or another type, it will be found out as the sequence data reveals this information (for instance HPV-16 sequence data is specific only for HPV-16 and a different sequence data would reveal this problem). Thus, accordingly to the invention the risk for false positive results would be minimal if with specific primer designs.

When a number of oligonucleotides are used together, they might interact together (hybridize to each other, so called primer-dimer) and get extended in the sequencing process in Pyrosequencing and may give rise to sequence signals. These signals may interfere with the DNA-sequencing. However, this problem could be circumvented if the non-hybridized primers are removed by an extra wash prior to Pyrosequencing and/or single strand binding protein (SSB) (Ronaghi, 2000), which inhibits primer dimer(s), cross hybridization of primers or non-specific hybridization, is used. For example, 1 μg of ssb is added to the reaction mix prior to Pyrosequencing.

Conditions allowing hybridization mean conditions wherein a type-specific primer is allowed to hybridize specifically to a type or types, without hybridization of non-type-specific primers. The skilled person may easily find out the right conditions. The sequencing may be performed by any sequencing technique. Preferably, conventional gel-based sequencing (Sanger dideoxy sequencing), sequencing-by-synthesis, sequencing by mass spectrometry or any other DNA-sequencing technologies that can apply the invention.

The DNA molecules could be used directly from genomic DNA for the method of invention if the DNA sequencing technology is sensitive enough to circumvent PCR.

In one embodiment of the present invention at least one primer is specific for a high-risk variant of a disease linked to the infectious microorganism. In another embodiment the microorganism is a human papillomavirus (HPV). Other applications are not excluded.

In one embodiment, the oligonucleotide primers of the kit are designed for being specific for any clinically/medically important human hapillomaviruses, preferably high-risk HPV-16, 18, 31, 33, 35, 39, 45, 51, 52, 58, 59, 66, 68, 69 and low-risk HPV-6, 11, 34, 40, 42, 43, 44.

Kits can be developed for specific typing and sequencing of microorganisms and viruses of interest or target DNA by DNA sequencing technologies. The kits could be used for genotyping/typing/identifying/identification and sequencing of phylum/class/order(subclass)/family/genus/species/subspecies/strains in microorganisms and viruses, or any other application requiring a multiple sequencing pool approach by DNA sequencing.

The invention will hereinafter be described by way of examples. These are not intended to limit the scope of the invention but merely to illustrate the invention.

### EXAMPLES

### Example 1

### Materials for HPV Typing

The primers for example 1 are designed for the HPV types that can be amplified with GP5+/6+ or MY09/11 primers sets. Each sequencing primer is designed to be specific for one designated HPV genotype. All the primers were checked by BLAST search for specificity and the resulting sequences were specific for the type designed.

The specific HPV-primers designed for detection of high-risk HPV by the multiple sequencing primer strategy of the invention were:

High-risk HPV genotypes

| | |
|---|---|
| HPV-16 | 5′-GCTGCCATATCTACTTCAGA |
| HPV-18 | 5′-GCTTCTACACAGTCTCCTGT |
| HPV-31 | 5′-GTG CTG CAA TTG CAA ACA GT |
| HPV-33 | 5′-ACACAAGTAACTAGTGACAG |
| HPV-45 | 5′-TATGTGCCTCTACACAAAAT |

Low-risk HPV genotypes

| | |
|---|---|
| HPV-6 | 5′-GTG CAT CCG TAA CTA CAT CTT |
| HPV-11 | 5′-GTG CAT CTG TGT CTA AAT CTG |

The GP5+/6+ amplification primer set sequence is:

| | |
|---|---|
| GP5+ | 5′-TTT GTT ACT GTG GTA GAT ACT AC 3′ |
| Biotin-GP6+ | 5′-GAA AAA TAA ACT GTA AAT CAT ATT C 3′ |

The MY09/11 amplification primer set sequence is:

| | |
|---|---|
| Biotin-MY09 | 5′-CGT CCM ARR GGA WAC TGA TC |
| MY11 | 5′-GCM CAG GGW CAT AAY AAT GG |

### Example 2

### Typing of HPV in Clinical Samples and Simulated Mixed-infections by Applying Multiple Sequencing Primers in Pyrosequencing Technology

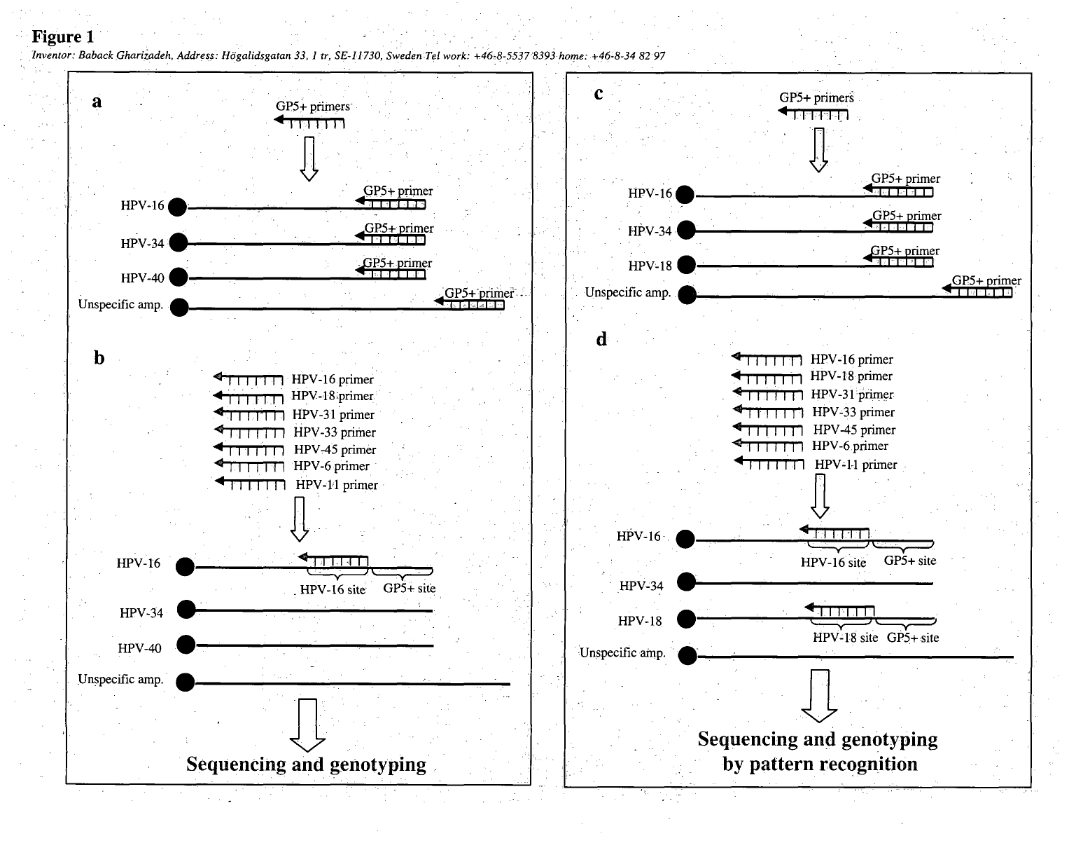

Some types of human papillomaviruses (HPV) are broadly recognized on a global scale as causative agents for cervical cancer. They are also related to other cancer types. For genotyping of HPVs we introduced the Pyrosequencing technology for specific HPV-typing (Gharizadeh et al., 2001). In general, not more than 25 bases are needed for specific genotyping. Multiple infections and unspecific amplification have been a problem for typing as double/multiple infections or unspecific amplification products present in one specimen give rise to sequence signals from all available types/products in the specimen. This is mainly because all HPV types in a sample are amplified with PCR utilizing the general GP5+/6+ primer set and then sequenced utilizing the GP5+ primer as extension/sequencing primer. This primer hybridizes to all present types, or unspecific amplified products, amplified by GP5+/GP6+ general primer set (FIGS. 1a and 1c). The principle of the new method of invention is illustrated in FIGS. 1b and 1d. Type-specific oligonucleotides for the most oncogenic (high-risk) HPV types (in the example; HPV-16, HPV-18, HPV-33 and HPV-45) are pooled and added to a mixed-infection (multi-variant) sample. If, the sample contains one of the four oncogenic HPV-types, one of the type-specific oligonucleotides will hybridize to that type (the oligonucleotides will not hybridize to low-risk HPV-types). After the hybridization the sample can be sequenced (with the hybridized oligonucleotide functioning as extension/sequencing primer), and the high-risk HPV-type can be detected and correctly typed.

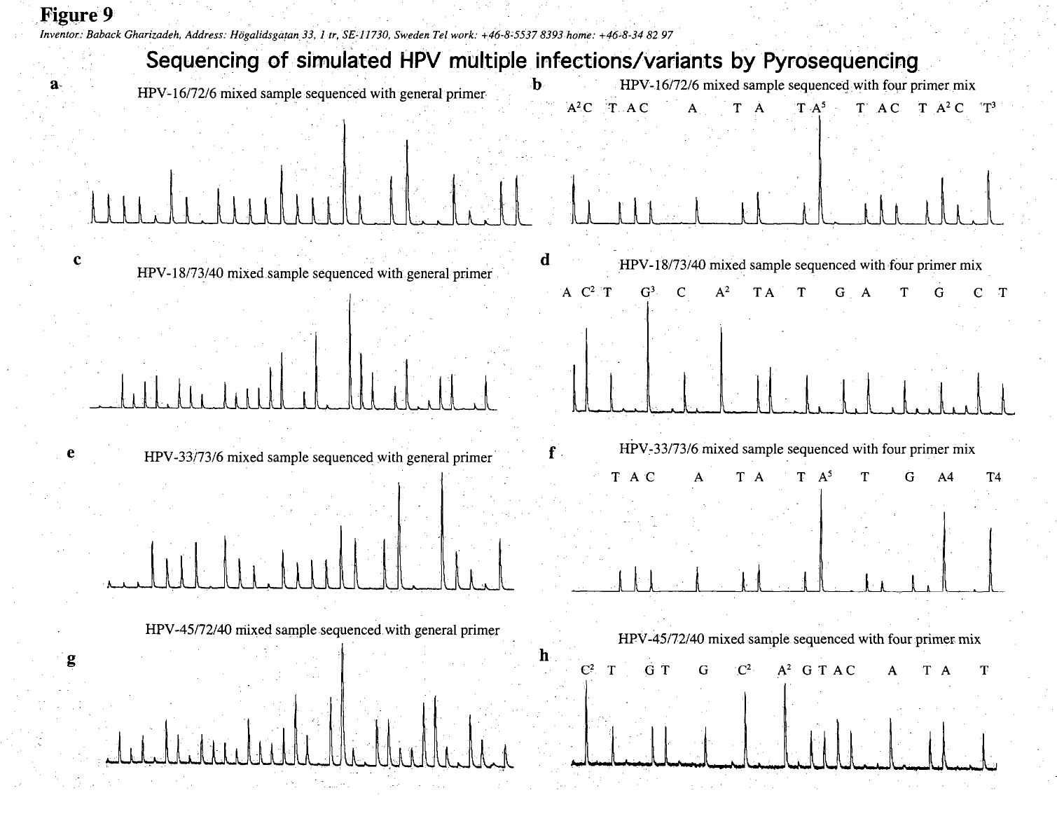

In this model system, type specific oligonucleotides were designed for detection of HPV-16, HPV-18, HPV-33 and HPV-45. Amplicons derived from HPV-6, HPV-16, HPV-18, HPV-33, HPV-40, HPV-45, HPV-72 and HPV-73 plasmid DNA (amplified with the general GP5+/6+ primer set), were mixed three and three in equal proportions (25 μl of each) prior to Pyrosequencing/dideoxy DNA-sequencing. Each triple-mix contained one high-risk and two low-risk types. After single-strand separation of PCR products, the primer hybridization step was performed on each PCR-mix in two separate reactions; one reaction was hybridized with the GP5+ primer and the other with the specific four-oligonucleotide-pool. The primed DNA samples were then sequenced 20-25 bases for genotyping with the Pyrosequencing technology. FIG. 9 shows typical traces from sequencing of the mixtures of HPV-16/72/6 (FIGS. 9a, 9b), HPV-18/73/40 (FIGS. 9c, 9d), HPV-33/73/6 FIGS. (9e, 9f) and HPV-45/72/40 (9g, 9h), sequenced with the GP5+ primer and the four-primer-pool in two different reactions. The triple mixtures primed with GP5+ (FIGS. 9a, 9c, 9e and 9g) indicate sequence signals from three types making genotyping almost impossible. On the other hand, the same triple mixtures primed with the four-oligonucleotide-pool (FIGS. 9b, 9d, 9f and 9h) indicate specific sequence signals and thereby allowing the correct genotype to be determined. The sequence data was analyzed with BLAST search, and for genotying of HPV-16, HPV-18, HPV-33 and HPV-45 no more than 18, 18, 20 and 17 bases, respectively, were needed. These experiments are described in detail (Gharizadeh et al., 2003).

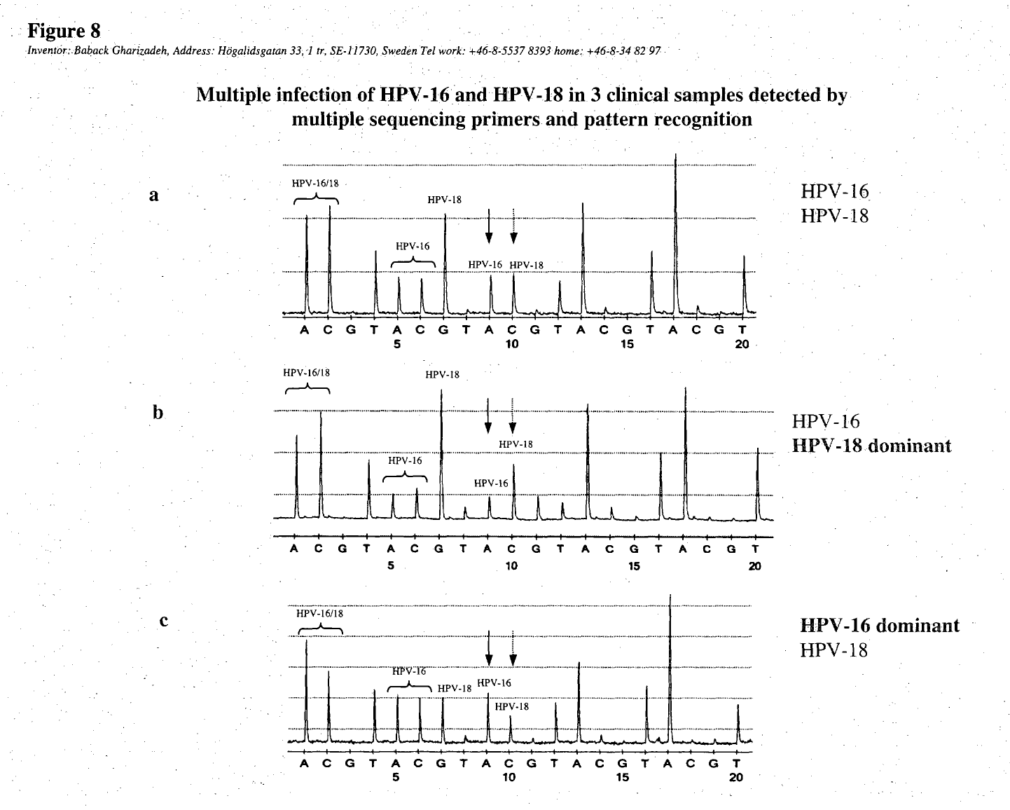

The same approach was also repeated on by a seven-primer-pool (specific for seven HPV genotypes) on a series of simulated multiple infections and satisfactory results were obtained (unpublished data). The seven-sequencing primer-pool was also applied for 80 clinical samples amplified separately by GP5+/6+ (150 bp fragments) and MY09/11 (450 bp fragments) primers sets. A substantial number of the samples contained multiple infections or unspecific amplification products, which were not typable by the GP5+ general sequencing primers (unpublished data). The multiple infections could by easily typed by pattern recognition. The dominance of each type could be easily observed in multiple infections. FIG. 8 shows three different clinical samples containing multiple infections. The multiple infections were genotyped by pattern recognition. As there are seven sequencing primers used in this approach, one characteristic pattern is reserved for each genotype. For example, HPV-18 is characterized by GGG in the 7^{th} nucleotide addition and HPV-16 is characterized by the peaks for A, C and A, in 5^{th} and 6^{th} and 9^{th} nucleotide addition order, respectively. FIG. 8 shows clearly these characterizations in HPV-16 and HPV-18 and the dominance of each genotype. HPV-16 and HPV-18 are the most common oncogenic genotypes that are found together in 70-80% cervical cancer specimens.

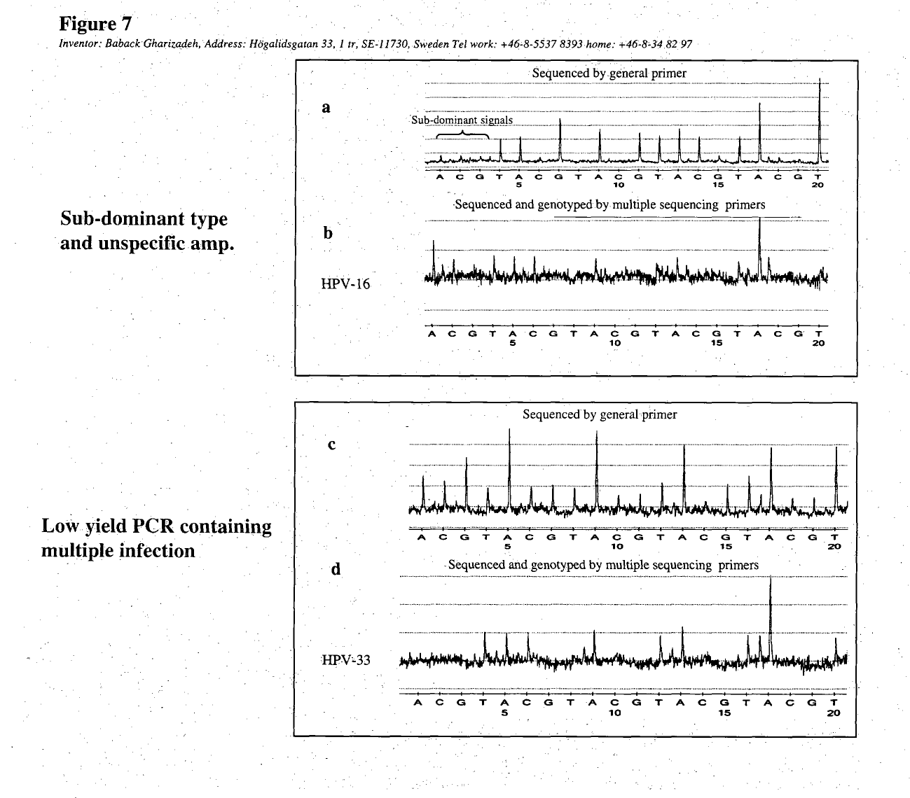

HPV genotype that was in minority compared to either multiple infection or unspecific amplification products were detected by multiple sequencing primer approach. FIG. 7a shows a clinical sample containing clinically important HPV-16, which is in minority and not detectable by the general primer due to high sequence signals from either unspecific amplification products or multiple infections and is genotyped by multiple sequencing primers despite its extremely low DNA concentration.

Amplicons with low PCR yield were also detectable and genotyped by the seven-multiple-sequencing primers. FIG. 7b shows the oncogenic HPV-33 in a clinical sample, which is not detectable because of low yield and is detected and genotyped by multiple-sequencing-primer.

Multiple sequencing primers for HPV genotyping were applicable to both MY09/11 and GP5+/6+ derived amplicons.

### Example 3

### Typing of HPV in Mixed-infections by Dideoxi DNA-sequencing (Sanger)

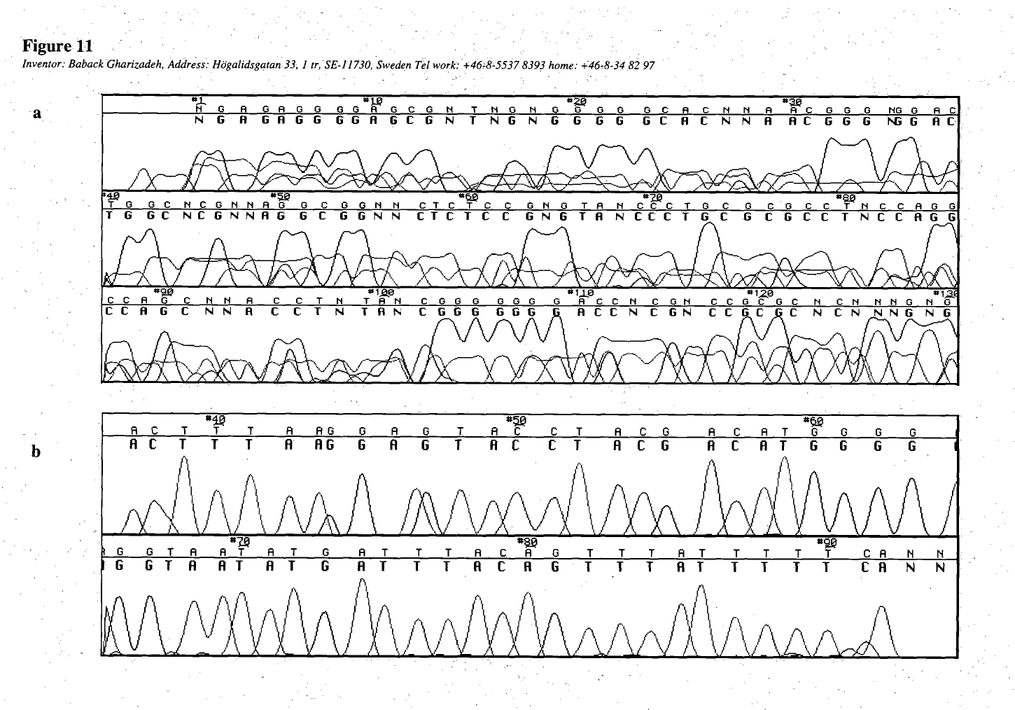

The general approach of example 2 was repeated, and Sanger sequencing was used as the DNA-sequencing method, instead of Pyrsosequencing (all the figures are not shown). FIG. 11a shows HPV-16/72/6-mixture sequenced with a general primer as extension primer. FIG. 11b shows HPV-16/72/6-mixture, sequenced by the primer set of the invention, which comprises a mix of 4 primers (HPV-16, 18, 33 and 45) (Gharizadeh et al., 2003). The results show clearly that with the dideoxy sequencing, typing with the primer set of the invention renders sequencing results that are clear to interpret than by using a general primer (impossible). An ABI prism DNA analyzer 3700 (Applied biosystems) was used for the DNA-sequencing with Big dye terminator kit according the manufacturer's manual. Same results were achieved on amplicons derived by MY09/11 using a-seven-multiple sequencing primers (unpublished data).

### Example 4

Amplicons containing unspecific amplification:

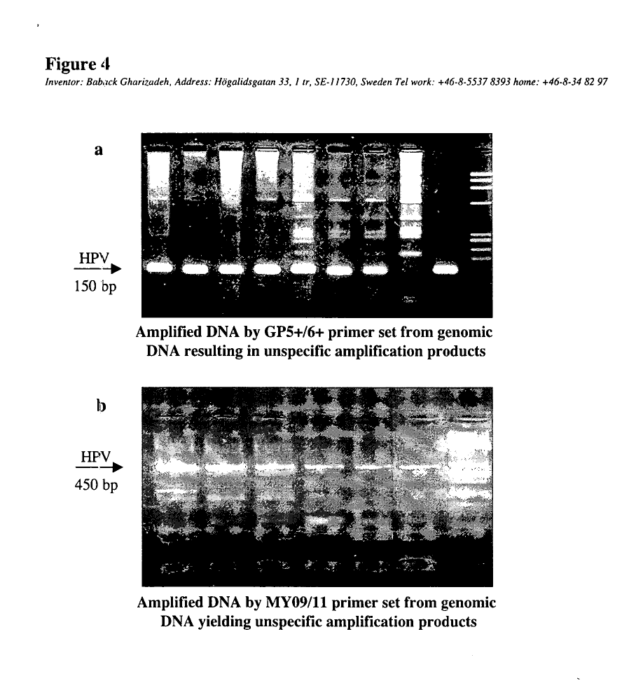

GP5+/6+ and MY09/11 primer sets tend to yield unspecific amplification products from genomic DNA (Fernandez-Contreras et al., 2000; Gharizadeh et al., 2001). FIG. 4 shows unspecific amplification products on agarose stained gels from amplicons derived from MY09/11 and GP5+/6+ primer sets. For this reason, HPV PCR is performed in a nested-PCR fashion for DNA sequencing, and in some cases, cloning is performed.

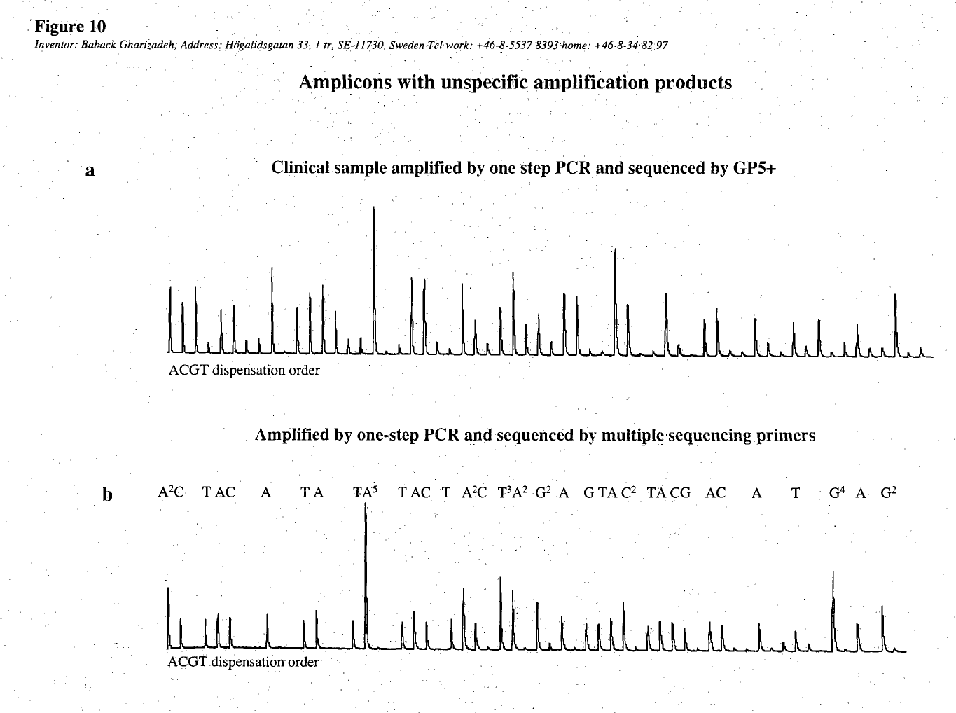

The GP5+ primer and the mulitple sequencing pool were used for sequencing of amplicons with one-step-PCRs containing unspecific amplification clinical samples. The GP5+ sequence data were unclear with signals from unspecific products. FIG. 8a shows unspecific sequence signals from HPV-16 amplicon primed with GP5+ (the same primer used in amplification), which make base calling and genotyping difficult. The sequence data from the muliple sequening primer pool from the same amplicons with unspecific amplification were clear and specific. FIG. 10b shows specific genotyping of HPV-16 using the primer pool. The same specimen was also amplified with a nested PCR using primer sets MY09/11 and GP5+/6+. As shown in FIG. 10b (compare with FIG. 10a), clear sequence signal peaks were acquired by nested PCR (Gharizadeh et al., 2003). Nested PCR or cloning could be avoided by using the muliple sequencing primer approach.

The same results were obtained on amplicons amplified directly by MY09/11. The majority of the samples contained unspecific amplification products when using MY09/11 PCR primer set. Good sequence resutls were obtained when using the multiple sequencing primer pool in the MY09/11-derived amplicons containing unspecific amplification products (unpublished data).

### Example 5

### Materials for Typing of Bacteria

Extension/sequencing primers:

16S Seq-primer 19b
GCTGGCACGTAGTTAGCCG
16S Seq-primer 31b
GTTAGCCGGTGCTTCTTCTG

By using the above mentioned primers the sequence data below is not necessary to sequence or analyze. In one group 19 bases is not necessary to sequence (compared to conventional technology) and in the other group 31 bases is not necessary to sequence. Here the sequences which could be skipped:

(1) GCTGGCACGTAGTTAGCCG
(2) GCTGGCACGGAGTTAGCCGGTGCTTCTTCTG

### Example 6

### Winning Read Length and Typing of Bacteria by Pyrosequencing Technology

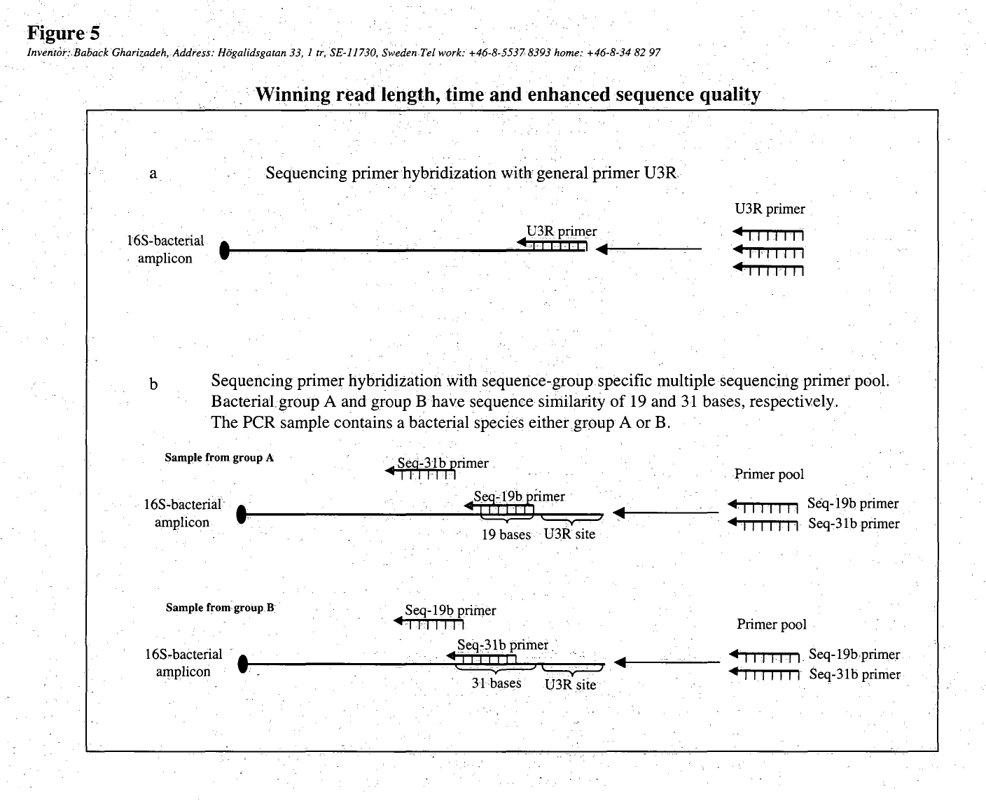

The 16S rRNA gene has traditionally been used for typing of bacteria. In the procedure the bacterial 16S rRNA gene is amplified by PCR with general primers binding to conservative regions on 16S rRNA gene. After single-strand separation, the immobilized single-strand DNA is primed with a general sequencing primer (e.g. U3R) (FIG. 5a). The obtained sequence data is analyzed on BLAST for identification. If the Pyroseuqencing technology is used for typing the data obtained might not be sufficient for correct typing. The reason is the limitation in read length obtained by Pyrosequencing technology. The first region close to the primer site is semi-conservative and thereby less informative. After this region a more variable region is reached, which is more useful for correct typing.

In the new method, the fact that microorganisms can be divided into groups, for which group-specific oligonucleotides can be designed, is utilized. The principle of the application is shown in FIG. 5b. Group-specific oligonucleotides are pooled and added to a bacterial DNA sample amplified with PCR utilizing general primers binding to conservative regions on the 16S rRNA gene. One of the group-specific oligonucleotides will hybridize with the DNA (dependant on which of the groups that are present). After the hybridization the sample can be sequenced and the bacterial type present in the sample can be detected and correctly typed.

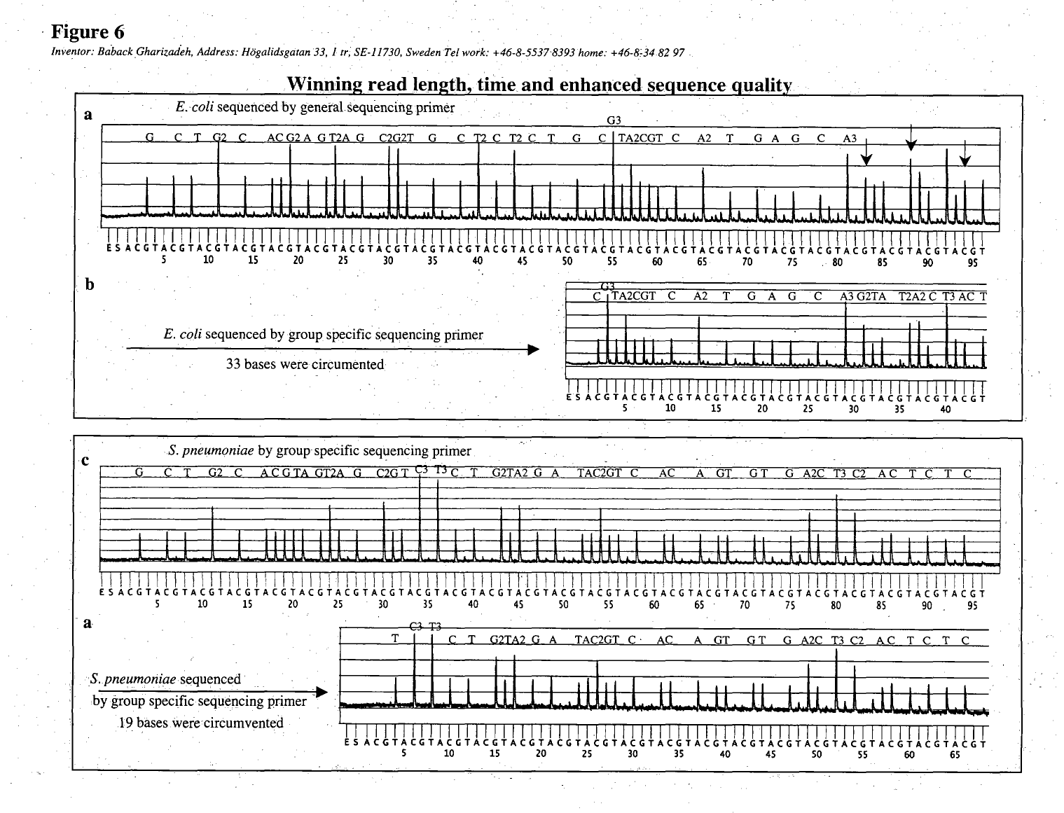

In a model system, group-specific oligonucleotides were designed for samples of bacteria that could be grouped in two categories; one group with the first 19 bases sequence similarity after the PCR primer and the second group with the first 31 bases in common. FIG. 6b shows pyrogram of Escherichia coli sequence data circumventing 31 bases by the oligonucleotide pool of invention. Bacterial 16S DNA was amplified with general primers Biotin U2F and U3R binding to conservative regions on 16S rRNA gene. After single strand separation, the immobilized single-strand DNA sample was hybridized with the pooled oligonucleotides. Fast and correct typing of bacteria from both groups was possible by the Pyrosequencing technology. Also in this study the sequencing data quality was improved. This was performed by Pyrosequencing technology. FIG. 6a shows an amplicon sequenced by the U3R general primer and sequence-group specific primers. The sequencing of 31 bases was circumvented and sequence quality was improved significantly by the multiple sequencing primers (FIG. 6b). FIGS. 6c and 6d show Streptococcus pneumoniae am licon sequenced by the U3R general primer and sequence-group specific primers. The sequencing of 19 bases was circumvented, making the DNA sequencing rapid and time-effective. This approach would be advantageous in DNA sequencing technologies when there is DNA read-length. This work is in press in journal of Molecular and Cellular Probes.

The bacteria family/species typed were Listeria species (such as Listeria moncyogenes), Staphyloococcus species (such as Staphyloococcus aureus, Staphyloococcus haemolyticus, Staphyloococcus pneumoniae), Streptococcus species (such as Streptococcus agalactiae, Streptococcus anginosus, Streptococcus intermedius, Streptococcus milleri, Streptococcus mitis, Streptococcus pneumoniae), Haemophilus species (such as Haemophilus influenzae), Neisseria species (such as Neisseria meningitidis), Enterococcus species (such as Enterococcusfecalis, Enterobacter cloacae) Escherichia coli and Klebsiella pneumoniae.

### Example 7

### Typing of Bacteria by Dideoxy DNA-sequencing

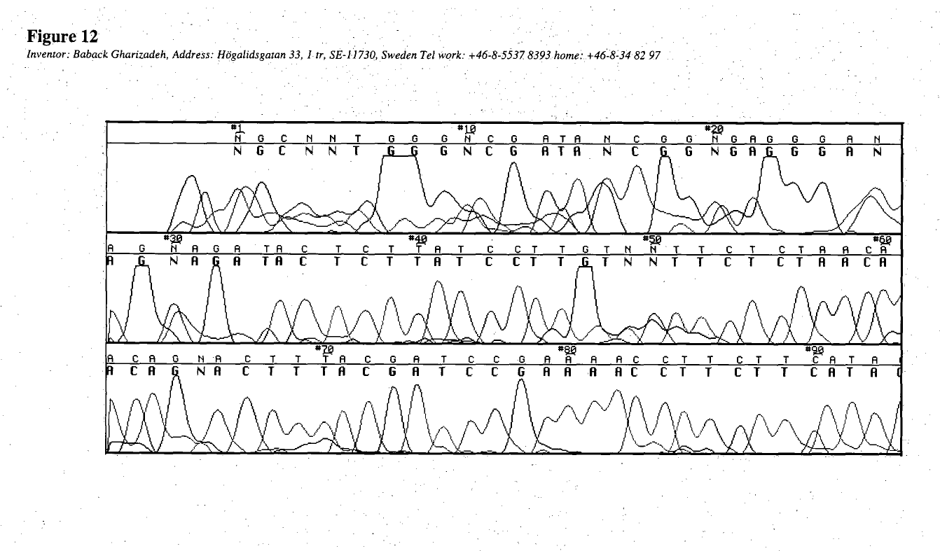

The general approach of example 6 was repeated, and dideoxy (Sanger) DNA-sequencing was used as the sequencing method. The primer pool comprising 19b/31b (19b binds to Listeria monocyogenes, 31b binds to Haemphilus influenzae. The results show that Sanger sequencing is also suitable to use for analysis of typing with the set of primers of the invention. FIG. 12 shows sequencing Listeria monocyogenes by sequencing primers of invention. An ABI prism DNA analyzer 3700 (Applied biosystems) was used for the DNA-sequencing with Big dye terminator kit according the manufacturer's manual. This work is in press in journal of Molecular and Cellular Probes.

### References

Berkenkamp, S., Kirpekar, F. and Hillenkamp, F.: Infrared MALDI mass spectrometry of large nucleic acids. Science 281 (1998) 260-262.
Fernandez-Contreras, M. E., Sarria, C., Nieto, S. and Lazo, P. A.: Amplification of human genomic sequences by human papillomaviruses universal consensus primers. J Virol Methods 87 (2000) 171-5.
Gharizadeh, B., Ghaderi, M., Donnelly, D., Amini, B., Wallin, K. L. and Nyren, P.: Multiple-primer DNA sequencing method. Electrophoresis 24 (2003) 1145-51.
Gharizadeh, B., Kalantari, M., Garcia, C. A., Johansson, B. and Nyren, P.: Typing of human papillomavirus by pyrosequencing. Lab Invest 81 (2001) 673-9.
Hagmar, B., Kalantari, M., Skyldberg, B., Moberger, B., Johansson, B., Walaas, L. and Warleby, B.: Human papillomavirus in cell samples from Stockholm Gynecologic Health Screening. Acta Cytol 39 (1995) 741-5.
Nordstrom, T., Nourizad, K., Ronaghi, M. and Nyren, P.: Method enabling pyrosequencing on double-stranded DNA. Anal Biochem 282 (2000a) 186-93.
Nordstrom, T., Ronaghi, M., Forsberg, L., de Faire, U., Morgenstern, R. and Nyren, P.: Direct analysis of single-nucleotide polymorphism on double-stranded DNA by pyrosequencing. Biotechnol Appl Biochem 31 (Pt 2) (2000b) 107-12.
Pourmand, N., Elahi, E., Davis, R. W. and Ronaghi, M.: Multiplex Pyrosequencing. Nucleic Acids Res 30 (2002) e31.
Ronaghi, M.: Improved performance of pyrosequencing using single-stranded DNA-binding protein. Anal Biochem 286 (2000) 282-8.
Ronaghi, M., Uhlen, M. and Nyren, P.: A sequencing method based on real-time pyrophosphate. Science 281 (1998) 363, 365.
Sanger, F., Nicklen, S. and Coulson, A. R.: DNA sequencing with chain-terminating inhibitors. Proc Natl Acad Sci USA 74 (1977) 5463-5467.
Skyldberg, B., Kalantari, M., Karki, M., Johansson, B., Hagmar, B. and Walaas, L.: Detection of human papillomavirus infection in tissue blocks by in situ hybridization as compared with a polymerase chain reaction procedure. Hum Pathol 22 (1991) 578-82.

1. The multiple sequencing oligonucleotide primer pool method is utilized for specific genotyping, typing, identification, detection and sequencing of a sample of nucleic acid molecules, whereby the molecules are suspected to contain at least one type or species or target of a variable region, region of interest or target DNA, each type having different nucleotide patterns, and the moiecuies are suspected to contain unspecific amplification in the amplification product and comprising the steps of:
A. Providing the sample is of nucleic acid molecules;
B. Providing a mixed set of at least two sequencing oligonucleotide primers, whereby each primer is designed for being specific for one type or species or group or target chosen from the known set of types or target of the nucleic acid sample, thereby allowing a primer, which is specific for a type, species, group or target that is present in the sample, to hybridize in or close to the target or variable region;
C. Mixing the set of sample and specific primers under conditions allowing a primer or primers to hybridize if a target type or types are present in the sample;
D. Determining the type, species or target region to which the primer or primers have hybridized by extending the hybridized primer or primers in a DNA sequencing reaction.

2. Method according to claim 1, wherein the sequencing reaction is performed by sequencing-by-synthesis, Sanger dideoxy sequencing, sequencing by mass spectrometry or any other DNA sequencing technology, applicable to the method of invention.

3. Method according to any one of claim 1-2, wherein the sample is a microorganism, such as a virus, fungi or bacteria.

4. Method according to any one of claim 1-3, wherein the sample is suspected to comprise at least two types, species or targets of nucleic acid molecules chosen from the known set of types, species or targets.

5. Method according to claim 4, wherein the sample contains multiple infection or variants or types or species.

6. Method according to claim 5, wherein at least one primer is specific for a variant of a disease linked to the microorganism.

7. Method according to claim 6, wherein the microorganism is a human papillomavirus

8. Method according to claim 7, wherein the known set of HPV-types are chosen from the group comprising the HPV-types; high-risk: 16, 18, 31, 33, 35, 39,45, 51, 52, 58, 59, 66, 68, 69 and low-risk: 6, 11, 34, 40, 42, 43, 44

9. Method according to any one of the preceding claims, wherein the sequence distance from the 3′-end of the general or consensus extendable primer could be avoided and to be instead sequenced in semi-conservative regions

10. Method according to any one of the preceding claims, for typing of amplicons comprising at least one semi-conservative region.

11. Method according to any one of claim 1-10 for typing of samples where a type or species is in minority or if the amplification fragment has low yield.

12. Method according to any one of claim 1-11, for typing of samples by DNA sequencing that contain unspecific amplification products, the primers of the set of primers not annealing to unspecific amplification products.

13. Kit for use in the method of typing of claim 1-12, comprising at least two oligonucleotide primers, whereby each primer is designed for being specific for one genotype or type or species or group or target or type-specific region chosen from a known set of types of the type-specific or target region of a nucleic acid sample, thereby allowing a primer, which is specific for a genotype or type or species or target that is present in the sample, to hybridize in or close to the type-specific or target region.

14. Kit according to claim 13, wherein the oligonucleotide primers of the kit are designed for being specific for any of the HPV-types chosen from the group comprising: high-risk: HPV-16, 18, 31, 33, 35, 39, 45, 51, 52, 58, 59, 66, 68, 69 and low-risk: HPV-6, 11, 34, 40, 42, 43, 44

15. Kit according to any one of the preceding claims for genotyping, typing, detection, identification or sequencing of any microorganisms, viruses or any other application where the multiple sequencing oligonucleotide primer pool approach is applicable by DNA sequencing technologies

16. Kit according to claim 1 for quantitative measurements of different genotypes or species or types amplified by PCR in the same sample or by mixing different amplicons
